(19) United States
(12) Patent Application Publication
(10) Pub. No.: US 2006/0159680 A1
(43) Pub. Date: Jul. 20, 2006

(54) Targeted apherisis for the treatment of rheumatoid arthritis

(76) Inventors: Henry J. Smith, Temecula, CA (US); James R. Smith, Laguna Niguel, CA (US)

Correspondence Address: STETINA BRUNDA GARRED & BRUCKER, 75 ENTERPRISE, SUITE 250, ALISO VIEJO, CA, 92656, US

(21) Appl. No.: 11/328,524

(22) Filed: Jan. 10, 2006

Related U.S. Application Data
(60) Provisional application No. 60/643,774, filed on Jan. 14, 2005.

(51) Int. Cl. A61K 39/395

(52) U.S. Cl. 424/140.1

(57) ABSTRACT

This invention uses “targeted apheresis” to treat rheumatoid arthritis patients. “Targeted Apheresis” is a process whereby the RF and immune complexes responsible for causing the disease symptoms are selectively removed from the blood by passing the blood through a cartridge containing immobilized IgG. The RF and immune complexes are bound out and the cleaned blood is returned to the patient Removal of circulating RF and immune complexes will ameliorate the symptoms of rheumatoid arthritis.

### CROSS-REFERENCE TO RELATED APPLICATIONS

This utility patent application claims priority to Provisional Patent Application Ser. No. 60/643,774, filed Jan. 14, 2005, entitled TARGETED APHERESIS FOR THE TREATMENT OF RHEUMATOID ARTHRITIS.

### BACKGROUND

The main application of this invention is in the treatment of immunological disorders such as rheumatoid arthritis and other inflammatory conditions. A common symptom of rheumatoid arthritis is swollen, painful joints. For mild cases of arthritis treatment usually consists of aspirin or non-steroidal anti-inflammatory drugs. For more severe cases steroidal drugs such as cortisone, prednisone and methylprednisolone are used. Finally, in cases where the patients become non-responsive to these drugs more cytotoxic drugs such as methotrexate may be used. In addition to their therapeutic effect these drugs all have a systemic effect and can cause serious side-reactions. It is desirable to have a treatment process that has fewer side effects.

Apheresis is a process whereby instead of treating the patient with drugs the patient's blood is passed through an extracorporeal device that removes the pathogenic substances that are causing the disease symptoms.

Rheumatoid arthritis patients have altered IgG in which “hidden” regions of the IgG molecule are exposed. They produce rheumatoid factor (RF) which is an IgM autoantibody that reacts with the altered IgG. This can result in the formation of immune complexes that can deposit in joints, organs and tissues to cause the symptoms of arthritis.

The current method of treating rheumatoid arthritis patients by apheresis utilizes an immunosorbent device to remove immune complexes. For example, the Prosorba Column is a single-use device that contains Protein A covalently bound to inert silica granules. When plasma is passed thru the device the immobilized Protein A binds out the circulating immune complexes. This process claims to remove about 750-1,500 mg of the circulating IgG-complexes from the patient's plasma. The cleaned plasma is then returned to the patient. This process however, is inefficient and removes native IgG along with the complexed IgG. Also, it does not remove the unbound RF autoantibodies which can still form immune complexes. There is also some controversy as to whether the observed beneficial effect is due to removal of immune complexes, or to the leaching out of small amounts of Protein A and other compounds which are introduced back into the patient.

It would be preferable therefore to develop a method that would be more efficient in selectively removing the immune complexes and RF autoantibodies involved in the inflammatory response.

This invention teaches a process of targeted apheresis that selectively removes the immune complexes and RF autoantibodies involved in the inflammatory response in rheumatoid arthritis.

### BRIEF SUMMARY

The main application of this invention is in the treatment of immunological disorders such as rheumatoid arthritis and other inflammatory conditions using “targeted apheresis”. “Targeted Apheresis” is a process whereby only the inflammatory substances causing the disease symptoms are selectively removed from the blood which is then returned to the individual. For treating rheumatoid arthritis, an immunosorbent apheresis cartridge containing immobilized human IgG is used to selectively remove immune complexes and RF from the blood.

### DETAILED DESCRIPTION

This invention describes a process of “Targeted Apheresis” that is used to selectively remove the immune complexes and RF from the blood of patients with rheumatoid arthritis. This represents a novel improvement over current methods of apheresis that remove a significant proportion of the patient's IgG immunoglobulins. This may have a deleterious effect upon the patient's ability to prevent infection and/or will stress the body to replace the lost immunoglobulins. Targeted apheresis avoids this by removing only the pathogenic autoimmune and inflammatory factors, leaving all the other blood elements intact.

Patients with rheumatoid arthritis develop autoantibodies against the “hidden region” of an altered IgG molecule. The autoantibody may be of the IgM class antibody (rheumatoid factor) or the autoantibody may be of the IgG class antibody. It is generally believed that the IgM rheumatoid factor is responsible for disease symptoms by combining with altered IgG to form immune complexes that deposit within joints and tissues.

Patients with active arthritis generally have elevated levels of RF. It is postulated that the arthritis patient for some reason produces altered IgG. The patient then develops an IgM autoantibody (RF) against the altered IgG. The binding of RF to altered IgG results in immune complex formation. There are a total of ten antigen binding sites on the RF IgM antibody molecule. However, because of the relatively lower levels of altered IgG not all of the IgM binding sites will be occupied. This invention postulates that arthritis patients have RF with free binding sites and immune complexes containing RF that also have free binding sites.

This invention teaches a process of targeted apheresis using immobilized altered IgG to selectively remove circulating RF and immune complexes from the blood of patients with rheumatoid arthritis.

Targeted apheresis utilizes the same apheresis equipment and procedure as conventional apheresis with one critical difference. The targeted apheresis cartridge employed is designed to selectively remove only the RF and immune complexes while leaving other blood components intact.

Preparation of the immobilized denatured IgG apheresis cartridge device

Purified IgG can be isolated from blood of humans and/or from different species of animals and used to prepare the apheresis cartridge device. RF has been shown to react with altered IgG from various species of animals. Altered IgG can be prepared by either heating the IgG fraction and/or by preparing antisera and then allowing the IgG antibodies to bind to the antigen thus exposing the “hidden” regions of the antibody molecule.

In the preferred embodiment of this invention human IgG is used to prepare the apheresis cartridge device. The IgG is isolated from human blood using standard laboratory methods. For example, human serum is treated with ammonium sulphate to salt out the immunoglobulin fraction which is further purified using gel-filtration, high pressure liquid chromatography and other laboratory methods. Alternatively, the IgG fraction is purified using the Cohn method of purification. These and other methods of purifying IgG are known to those skilled in the art and are within the scope of this invention.

The purified IgG is heat denatured by heating at 60° C. for 15 minutes to expose the hidden regions of the molecule. Other methods of exposing the hidden regions of the molecule may be employed that are known to those skilled in the art and are within the scope of this invention.

The altered IgG is immobilized by chemically coupling it to an insoluble support matrix such as agarose beads. For example, agarose beads are activated using cyanogen bromide and the IgG is incubated with the activated agarose to allow coupling to occur. The unconjugated material is removed by washing with buffer and the IgG bound agarose is packed into the targeted apheresis device. There are many different methods of chemically coupling proteins to a variety of insoluble support matrixes. These matrix materials and methods of protein coupling are known to those skilled in the art and are within the scope of this invention.

Typically, the apheresis device will be constructed as a cylinder with an inlet to allow plasma to enter at one end, and an outlet at the opposite end to allow the cleaned plasma to exit and be returned to the patient. Other device configurations may also be designed and are within the scope of this invention.

The cartridge device is constructed of material that is nontoxic and which provides rigid support to the agarose within. Typically, the material will be a plastic composition such as polystyrene, or polyvinyl, or polypropylene or polycarbonate or other similar material. There is an inside filter at the bottom of the device to prevent the agarose beads from leaving the device. There is also an inside filter at the top of the device to contain the agarose within the device. Typically these filters are composed of plastic and/or cellulosic material and have pores that will allow thru passage of fluid such as plasma, but not particulate material such as agarose beads. The manufacture of these types of devices and the materials used are known to those skilled in the art and are within the scope of this invention.

Apheresis procedure using immobilized denatured IgG

The overall procedure for targeted apheresis is the same as that used in conventional apheresis. Briefly, blood from the patient is circulated extra corporeally using standard apheresis equipment. The blood is separated into the cellular elements (red blood cells, white blood cells and platelets) and fluid (plasma) elements using differential centrifugation or a membrane filter. The plasma is then pumped through the targeted apheresis device where the RF and RF complexes will bind to the immobilized IgG. The cleaned plasma is then mixed with the cellular blood elements and returned to the patient.

The targeted apheresis cartridge may be employed as a single use device or it may be regenerated and used multiple times. To regenerate the device an elution buffer solution is passed through the device to release the RF and immune complexes bound to the immobilized IgG. For example, an elution buffer such as glycine-HCl buffer pH 2 will dissociate antigen: antibody bonds. The unbound antigen is washed out of the device and the regenerated antibody-agarose matrix is then washed and stored in physiological buffer such as phosphate buffered saline pH 7.2 with preservatives. Other similar eluting buffers and storage buffers are known to those skilled in the art and are within the scope of this invention. Typically, the cartridge device is stored in the cold at 2-8 C.

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A method of using targeted apheresis to treat rheumatoid arthritis.

2. A method according to claim 1 whereby the process of targeted apheresis utilizes a device containing immobilized animal IgG antibody.

3. A method according to claim 1 whereby the process of targeted apheresis utilizes a device containing immobilized human IgG antibody.

4. A method according to claim 3 where the antibody is conjugated to an agarose support matrix or similar support matrix.

5. A method according to claim 3 wherein the device is constructed as a rigid container using polystyrene, polypropylene, polycarbonate or other similar material, and wherein the device comprises an inlet aperture and an outlet aperture.

6. A method according to claim 3 where the device is a disposable device for single use only.

7. A method according to claim 3 where the device is regenerated and used multiple times.
